(19) United States
(12) Patent Application Publication
(10) Pub. No.: US 2012/0164185 A1
(43) Pub. Date: Jun. 28, 2012

(54) ORAL NUTRIENT OR MEDICANT CARRIER HAVING INCREASED SURFACE AREA AND ABSORPTION RATE

(76) Inventor: Barry Markman, Las Vegas, NV (US)

(21) Appl. No.: 13/339,029

(22) Filed: Dec. 28, 2011

Related U.S. Application Data
(60) Provisional application No. 61/427,642, filed on Dec. 28, 2010.

(51) Int. Cl. A61K 9/00; A61P 3/02; B82Y 30/00; B82Y 5/00

(52) U.S. Cl. 424/400; 977/773; 977/906

(57) ABSTRACT

A nutrient carrier comprises a body which is particularly suited for delivering nutrients via the oral cavity and pharynx. The body of the nutrient carrier comprises or includes a nutrient carrier element or material/composition. For example, the nutrient carrier body may be constructed from a nutrient carrying composition. In other embodiments, the nutrient carrier body may be constructed from a carrier material which carries a desired nutrient (such as a supplement, vitamin, mineral or medication/drug).

### RELATED APPLICATION DATA

This application claims priority to U.S. Provisional Application Ser. No. 61/427,642, filed Dec. 28, 2010.

### FIELD OF THE INVENTION

The present invention is in the field of a novel nutrient delivery carrier that is particularly suited for the oral cavity and pharynx.

### BACKGROUND OF THE INVENTION

Drug absorption and bioavailability is the outcome of a complex chain of events, and is among others influenced by the drug's solubility, permeability through the gastrointestinal wall, and its first pass gut wall and liver metabolism. Excluding liver metabolism, all other factors are characterized by the term oral absorption.

Furthermore, the delivery of nutrients, supplements, and medicine into the bloodstream through the oral cavity, which can be characterized by the anatomical regions inside the mouth (buccal, alveolar, sublingual, tonsilar, and palatine areas) and the superior and hypo-pharynx.

Different parts of oral cavity tissue have different degrees of permeability. The higher permeability capacity of a tissue, the better this tissue can absorb liquids. Sublingual mucosa is the thinnest among mouth tissues, and has the highest permeability capacity and the buccal and alveolar cavity in the mouth has the largest capacity to retain nutrients and medications. Compared to other ways of a drug supplement intake, oral cavity absorption has a number of benefits. First, this method is much quicker than other methods. Drug impact is measured by its concentration in blood. When a drug is swallowed, it can take hours before the desired concentration in blood is reached. In case of the oral cavity and pharynx pathway it can take just minutes and it allows the drug to bypass a digestive system. Some substances, when swallowed, can be degraded before they reach the bloodstream. This can happen in the stomach, small intestine, or in the liver. The oral cavity path takes a drug directly into the bloodstream.

Another case when the oral cavity method can help is when a person cannot digest some specific nutrient because of a medical condition.

Another advantage is that the method is more acceptable than other delivery methods. In cases when a relatively high concentration of key elements of a supplement, vitamin, mineral, or drug cannot be swallowed, a usual way is to prescribe injections. Since a significant number of medications can be absorbed without swallowing, the oral cavity and pharynx route can be beneficial.

There are several factors which determine how substances (drug, vitamins or minerals) are absorbed inside the mouth. Fat-soluble drugs are absorbed better when taken by means of the oral cavity and pharynx than are water-soluble ones. Tissues inside the mouth, as any body tissue, consist of cells, and the cell membrane is lipoidal (fat-like). Because of this molecules of materials which are better soluble in fat can penetrate cell membranes more easily. Small molecules penetrate through oral tissue better than larger molecules. Molecules penetrate better than ions (an ion is a charged molecule, or atom). Based on the solubility factor, it seems that water-soluble vitamins (vitamins C, H, B complex) would not be absorbed as well as fat-soluble vitamins (vitamins A, D, E, K).

Recent studies show that small molecules (molecular weight less than 100) cross the oral mucosa easily (molecular weights of all vitamins are greater than 100: vitamin C has the smallest weight of 176 and vitamin B12 has the highest molecular weight of 1357).

There are vitamins with neutral molecules, such as vitamins B3 and B12. Others have positively charged molecules—vitamin B1 (thiamine), and some vitamin molecules carry negative potential—vitamin C (ascorbic acid) or B2 phosphate. As neutral molecules can be absorbed better than charged molecules (ions), it seems that some vitamins would be absorbed more easily in the mouth than would others.

It turns out there are some limits as far as the amount of active ingredients a single strip can carry. In the past, dissolvable strips for vitamin delivery were unsuccessful due to technical restrictions. The limit for the active components is about 30% of the total weight of the current oral strips. A typical 120 mg film strip can only deliver about 36 mg of vitamin supplements. To put this number in perspective, a daily value (DV) of vitamin C is 60 mg, of vitamin E—20 mg, vitamin B3 (niacin)—20 mg, pantothenic acid—10 mg. Obviously, several strips would be required to provide the daily value concentration of required vitamins.

### SUMMARY OF THE INVENTION

One aspect of the invention is a novel nutrient delivery carrier that is particularly suited for the oral cavity and pharynx. Preferably, the invention comprises a carrier which is designed to increase the surface area exposure to mucosa of the oral cavity and pharynx.

In one embodiment, the nutrient carrier comprises a physical body which has a shape and/or other physical characteristics which are designed to maximize delivery to the anatomical regions inside the mouth. Preferably, the shape or other configuration of the carrier is selected to increase the surface area exposure to saliva in the mouth to allow for increased concentrations and rapid dissolving and absorption of the nutrient/medicant. For example, the body may have perforations, corrugations, invaginations and/or various projections which: (1) increase the effective surface area of the carrier and/or (2) contour to and thus increase the contact area of the carrier relative to saliva and/or anatomical regions in the mouth.

In a preferred embodiment, the nutrient carrier body comprises or includes a nutrient carrier or nutrient composition. Such a composition may comprise various combinations of supplements, vitamins, minerals, medications for use in humans and animals.

Advantageously, the nutrient carrier comprises a formulation that rapidly dissolves, is contoured to enhance ease of use and increase surface area, and is synergistic with an active ingredient for treating the indicated ailment, with the primary result of increasing the bioavailability of the active ingredients into the bloodstream and targeting capabilities to enhance the ingredients therapeutic effect.

Further objects, features, and advantages of the present invention over the prior art will become apparent from the detailed description of the drawings which follows, when considered with the attached figures.

### DESCRIPTION OF THE DRAWINGS

FIG. 1 illustrates one embodiment of a nutrient carrier in accordance with the invention, the carrier comprising a body having a hollow cylinder shape with perforations to increase surface area and absorption potential in the sublingual and buccal area;

FIG. 2 illustrates another embodiment of a nutrient carrier in accordance with the invention, the carrier comprising a body having a customized shape and including perforations to increase surface area and absorption potential in the Palatine area; and

FIG. 3 is a lateral view of the nutrient carrier illustrated in FIG. 2 showing various projections on the nutrient carrier body to increase surface area and absorption potential.

### DETAILED DESCRIPTION OF THE INVENTION

In the following description, numerous specific details are set forth in order to provide a more thorough description of the present invention. It will be apparent, however, to one skilled in the art, that the present invention may be practiced without these specific details. In other instances, well-known features have not been described in detail so as not to obscure the invention.

One embodiment of the invention comprises a water-erodible pharmaceutical carrier device. In one preferred embodiment, the carrier device comprises a multi-layered device which, when applied to mucosal surfaces, adheres immediately and delivers actives to treat specific localized diseases/disorders.

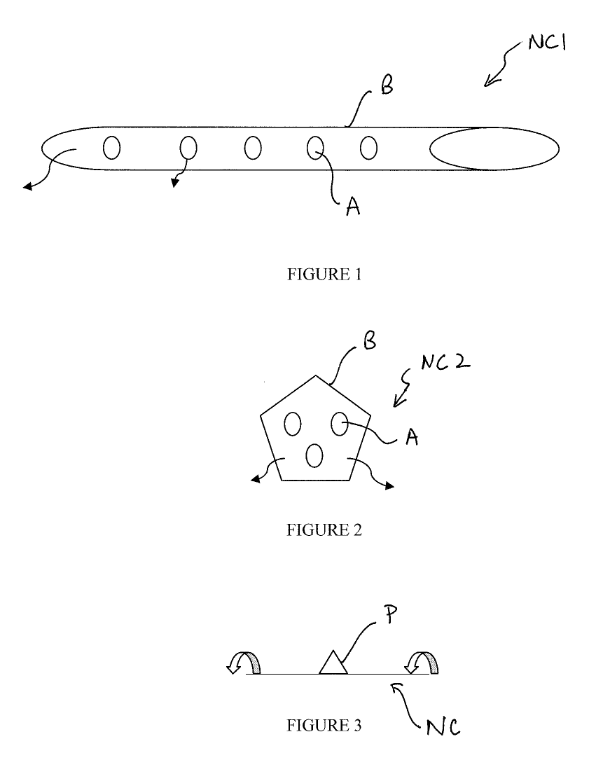

FIGS. 1-3 illustrate various embodiments of nutrient carriers in accordance with the present invention. Preferably, the nutrient carriers comprise bodies which are physically configured to maximize delivery to the anatomical regions inside the mouth, such as based upon the shape of the body and/or one or more other characteristics of the body, such as perforations.

Preferably, the shape or other configuration of the carrier is selected to increase the surface area exposure to saliva in the mouth to allow for increased concentrations and rapid dissolving and absorption of the nutrient/medicant. For example, the body may have perforations, corrugations, invaginations and/or various projections which: (1) increase the effective surface area of the carrier (especially relative to a given sized carrier—whereby the surface area is increased without causing the overall size of the carrier to be undesirably increased) and/or (2) contour to and thus increase the contact area of the carrier relative to saliva and/or anatomical regions in the mouth.

For example, FIG. 1 illustrates one embodiment of a nutrient carrier NC1 comprising a body B having a hollow cylinder shape with perforations or apertures A to increase surface area and absorption potential in the sublingual and buccal area.

FIG. 2 illustrates another embodiment of a nutrient carrier NC2. This carrier comprises a body B having a customized shape and includes perforations or apertures A to increase surface area and absorption potential in the Palatine area.

As illustrated in FIG. 3, the nutrient carrier NC may comprises a body which includes one or more projections P to increase surface area and absorption potential. The number and shape of the projections P may vary. As illustrated, the projections may be utilized with a generally planar nutrient carrier (and be associated with one or both sides thereof), or may be used with non-planar nutrient carriers such as that illustrated in FIG. 1.

In one embodiment, the body of the nutrient carrier comprises or includes a nutrient carrier element or material/composition. For example, the nutrient carrier body may be constructed from a nutrient carrying composition. Such a composition may comprise a carrier material combined with the desired nutrient (such as a supplement, vitamin, mineral and/or medication/drug). In other embodiments, the nutrient carrier body may be constructed from a carrier material and the body may carry the desired nutrient (such as a supplement, vitamin, mineral or medication/drug), such as in a space defined by the body.

Preferably, the nutrient carrier material or the composition thereof is also (along with the shape/configuration of the nutrient carrier body) selected to maximize delivery to the anatomical regions of the mouth. In particular, the present invention comprises nutrient carriers that are not only synergistic with the combination of active ingredients, but pertains to nutrient carriers that maximize the advantages of primary absorption in the oral cavity and pharynx.

In one embodiment, dark chocolate and/or cocoa powder is utilized as the nutrient carrier material. For example, the nutrient carrier may comprise a body having the form of a thin wafer of a dark cocoa material. In this configuration, the nutrient carrier is rapidly dissolving, contains trans-mucosal absorption enhancing agents, is contoured for the oral cavity and may be perforated to increase total surface area, and has a high concentration of total antioxidants. The desired nutrients may be located inside the nutrient carrier body or may be incorporated into the cocoa material to form a composition from which the body is constructed.

Preferably, the present invention increases the oxidation reduction absorption capacity (ORAC) of the carrier and the in vivo bioavailability of antioxidant dissolving formulation into the bloodstream and to the target organ. A well documented formulation for macular degeneration in soft gel form contains Lutein, vitamin C, E, Omega 3, zinc and copper. In one embodiment, such materials are used in combination with the antioxidants in dark chocolate and/or cocoa, whereby the materials are synergistic. The desired nutrients may be located inside the nutrient carrier body or may be incorporated into such materials to form a composition from which the body is constructed.

In another example, the nutrient carrier includes or comprises vitamin D, whereby by nutrient carrier includes the therapeutic effects vitamin D on certain carcinomas are obtained (such as in combination with the therapeutic effects of antioxidants in dark chocolate and/or cocoa on certain carcinomas). The desired nutrients may be located inside the nutrient carrier body or may be incorporated into such a material to form a composition from which the body is constructed.

In yet another example, the nutrient carrier includes or comprises vitamin C, whereby the therapeutic effects of vitamin C on respiratory infections are obtained (such as in combination with the enhanced antioxidant concentration in the dark chocolate and/or cocoa). The desired nutrients may be located inside the nutrient carrier body or may be incorporated into such a material to form a composition from which the body is constructed.

Dark chocolate is not the only nutrient carrier with beneficial therapeutic effects, but the nutrient carrier may include dissolvable green tea and grape seed formulations. In additional, various plant derivatives are excellent sources of vitamin C that can be not only dissolvable nutrient carriers but also an active ingredient, including but not limited to plant sources such as (in amounts of mg/100 g): Kakadu Plum (3100), Camu Camu (2800), and/or Rose Hip (2000). The desired nutrients may be located inside the nutrient carrier body or may be incorporated into such a material to form a composition from which the body is constructed.

Trans-mucosal absorption is critical in providing peak concentrations of the active medicants over time (T max), the period of time that the medicants levels are in the bloodstream (C max), and the average total concentration of medicant during this time period (Area Under the Curve-AUC). However, the therapeutic effect of the medicant can be enhanced by integrating a targeting capability of the medicant to the specific organ within the body. For example, flavonoids (both flavonols and flavanols) are most commonly known for their antioxidant activity in vitro. The antioxidant abilities of flavonoids in vitro are higher than those in vitamin C and E.

The present invention also pertains to enhancing the therapeutic effect and bioequivalence of the active ingredients by incorporating in vivo targeting molecules in the trans-mucosal nutrient carrier.

In one embodiment, the nutrient carrying composition may be processed to reduce the particle size of the material, such as to micron or nano-particle size.

Existing OraDisc™ technology can deliver drugs systemically across the mucosal surface or for the release of drugs into the oral cavity. The present invention does not rely on adhesion to the mucosal and a multi-layered device, but rather rapid absorption, increased surface area through perforations and patterns, contours designed to specific areas of the oral cavity, and/or molecules that enhance trans-mucosal of both carrier and active ingredients in the carrier.

Other embodiments of the invention comprise methods of manufacturing or making a nutrient carrier in accordance with the present invention. Such methods may include the use of various manufacturing equipment, including computer controlled equipment. Such equipment may thus include computer readable code or “software” which is used to control the equipment.

It will be understood that the above described arrangements of apparatus and the method there from are merely illustrative of applications of the principles of this invention and many other embodiments and modifications may be made without departing from the spirit and scope of the invention as defined in the claims.

What is claimed is:

1. A nutrient carrier device comprising:
a body which is physically configured to maximize delivery to the anatomical regions inside the mouth of a human or animal, said body having at least one perforation, corrugation, invagination or projection to increase and effective surface area thereof, and said body comprising a nutrient carrying composition comprising a nutrient carrier and a nutrient comprising one or more of a supplement, a vitamin, a mineral and a medication.

2. The nutrient carrier device in accordance with claim 1 wherein said composition comprises a plant derived material that is dissolvable in the oral cavity or pharynx and which includes at least one supplement, vitamin, mineral or medication.

3. The nutrient carrier device in accordance with claim 1 wherein said composition comprises a plant derived antioxidant material that is dissolvable in the oral cavity or pharynx and which includes at least one supplement, vitamin, mineral, medication or additional antioxidant material.

4. The nutrient carrier device in accordance with claim 1 wherein said composition comprises a cocoa derived antioxidant material that is dissolvable in the oral cavity or pharynx and which includes at least one supplement, vitamin, mineral, medication or additional antioxidant material.

5. The nutrient carrier device in accordance with claim 1 wherein said composition comprises an acyl berry material which is dissolvable in the oral cavity or pharynx and which includes at least one supplement, vitamin, mineral, medication or additional antioxidant material.

6. The nutrient carrier device in accordance with claim 1 wherein said composition comprises a green tea material which is dissolvable in the oral cavity or pharynx and which includes at least one supplement, vitamin, mineral, medication or additional antioxidant material.

7. The nutrient carrier device in accordance with claim 1 wherein said composition comprises a synthetic antioxidant material which is dissolvable in the oral cavity or pharynx and which includes at least one supplement, vitamin, mineral, medication or additional antioxidant material.

8. The nutrient carrier device in accordance with claim 1 wherein said composition comprises a material which increases the amount and speed for dissolvability and absorption into the bloodstream.

9. The nutrient carrier device in accordance with claim 1 wherein said composition comprises a material having one or more ions to increase dissolvability and absorption into the bloodstream.

10. The nutrient carrier device in accordance with claim 1 wherein said composition comprises ranges of low and high pH water, various ranges of water cluster sizes, and various ranges of positive and negative ionic potentials to increase dissolvability and absorption into the bloodstream.

11. The nutrient carrier device in accordance with claim 1 wherein said composition contains various ingredients to enhance taste, vary viscosity, and consistency without decreasing the efficacy of the antioxidants, their dissolvability, and absorption into the blood stream.

12. The nutrient carrier device in accordance with claim 1 wherein said body is configured to increase the conformity to the different surface areas of the oral cavity to increase dissolvability and absorption into the bloodstream.

13. The nutrient carrier device in accordance with claim 1 wherein said body comprises a generally planar body which has at least one perforation, corrugation, invagination or projection to increase the surface area, dissolvability, and absorption into the bloodstream.

14. The nutrient carrier device in accordance with claim 1 wherein said composition comprises a material which has been reduced in particle size.

15. The nutrient carrier device in accordance with claim 14 wherein said materials have been reduced to nano particle size.

16. The nutrient carrier device in accordance with claim 14 wherein said materials have been reduced to micron particle size.
